(19) United States
(12) Patent Application Publication
(10) Pub. No.: US 2024/0138708 A1
(43) Pub. Date: May 2, 2024

(54) PRESSURE SENSING BRACE AND METHODS OF USING SAME

(71) Applicant: SE Watson & Associates, LLC, Holden Beach, NC (US)

(72) Inventor: Stephen E. Watson, Holden Beach, NC (US)

(21) Appl. No.: 18/385,278

(22) Filed: Oct. 30, 2023

Related U.S. Application Data
(60) Provisional application No. 63/421,439, filed on Nov. 1, 2022.
(60) Provisional application No. 63/465,135, filed on May 9, 2023.

(51) Int. Cl. A61B 5/11; A61B 5/00

(52) U.S. Cl. CPC A61B 5/1107; A61B 5/0022; A61B 5/4542; A61B 5/4576; A61B 5/458; A61B 5/459; A61B 5/6812; A61B 5/6831; A61B 5/742; A61B 5/746; A61B 5/6823; A61B 5/6824; A61B 5/6828; A61B 2560/0406; A61B 2560/045; A61B 2560/0462; A61B 2562/0219; A61B 2562/0247; A61B 2562/164

(57) ABSTRACT

A pressure sensing brace includes a strap having two sides, one side configured to be positioned over and removably fastened to a body surface over a preselected body muscle. The brace further includes a pressure sensor integrated with the strap and configured to detect changes in pressure applied to said one side of said strap in response to a change in the status of said preselected body muscle from a relaxed state to a flexed state, and vice versa. The pressure sensing brace can include a haptic feedback module, visual indicators, motion sensors, and other forms of indicators that will provide a means when the strap experiences a certain level of pressure. The brace can provide feedback allowing a user to monitor the state of a body part involved in a certain activity so the user can adjust their effort level based upon the tension in the body part.

### CROSS-REFERENCE TO RELATED PATENT APPLICATIONS

This U.S. non-Provisional Patent Application claims priority to and the benefit of U.S. Provisional Patent Application No. 63/421,439, filed on Nov. 1, 2022, the entirety of which is incorporated herein by reference. Furthermore, this U.S. non-Provisional Patent Application similarly incorporates by reference the entirety of U.S. Provisional Patent Application No. 63/465,135, filed on May 9, 2023, herein.

### BACKGROUND

Elbow tendinopathy is commonly known as either tennis elbow (lateral epicondylitis) or golfer's elbow (medial epicondylitis). These tendinopathies result from overload injuries either acutely or chronically, with repetitive forces. Elbow epicondylitis is a common ailment in the population effecting roughly 3 percent of the general population aged 35-65. Common professions include musicians, chefs, manual laborers, and athletes (e.g., racquet sports, golfers and baseball players).

Tendinopathy is the result of a micro-tearing of the tendon at the insertion to the bone. The pathophysiology of tendinopathy includes tendon cellular and matrix changes with alterations in nociceptive processing impairing motor and sensory function. Tendinopathy has increased cellularity, accumulation of ground substance, abnormal arrangement of collagen fibers and neurovascular growth leading to reactive tendinopathy or degenerative tendinopathy which may lead to focal necrosis or calcification. The collagen in the tendon bone interface losses it's strength with repetitive injuries. The tendon becomes fragile and can break, causing the body to form scar tissue causing the tendon to thicken. The common tendon affected for lateral epicondylitis is the extensor carpi radialus brevis. The common tendons for medial epicondylitis are the flexi carpi ulnaris and pronator teres. Patients typically experience pain and reduced function with activities of daily living on either the medial or lateral side of the elbow.

Difficulties in managing elbow epicondylitis are a result of the complexity of the anatomy, biomechanics and pathophysiology. Numerous treatment options are available. Ninety five percent of the patients respond to conservative (non-operative) management. Common treatments include rest, medication (NSAIDS and steroid injections), activity modification, physical therapy, and counterforce bracing. Conventional bracing methods use counterforce straps. Current elbow strap technology is limited by an inability to provide tension feedback for proper bracing adjustment resulting in ineffective use if applied with too little tension, or the potential for bodily harm if excessively tensioned. Current technology also lacks the ability to detect activity in the upper extremity that can perpetuate the tendinopathy. Therefore, a need exists for a device and method to properly apply a counterforce strap. Secondly, a need exists to properly monitor excessive contraction of the forearm musculature which creates a detrimental physiological state.

In addition, there is a need for a device and method of measuring the amount of strain on a body part or muscle group when performing particular tasks that are not directly related to treating the conditions detailed above. Proprioception refers to a body's sense of self-movement, force, and body position. Proprioception is mediated by proprioceptors, mechanosensory neurons located within muscles, tendons, and joints. It is possible, if not probable, that people tend to tense or move a body part specifically to be aware of the location and position of that body part.

Awareness of the body's position and movement is a trait that will inevitably vary from one person to another. It is likely that people that are particularly skilled at certain tasks have a higher level of proprioceptive awareness allowing them to be able to perform tasks and make them appear effortless. For example, guitar players have a natural tendency to tense the hand and forearm they use to manipulate the fretboard, but many world class guitar players appear to play with very relaxed hands and forearms. It is likely that different levels of proprioceptive awareness dictate (at least in part) muscular tension, and how well people perform certain tasks.

### SUMMARY OF THE INVENTION

According to a first aspect, a bracing system for indicating a potential strain to a preselected body muscle includes a strap having two sides, one side configured to be positioned over and removably fastened to a body surface directly over a preselected body muscle. The system also includes a pressure sensor coupled to or integrated with the strap and configured to detect changes in pressure applied to said one side of said strap in response to a change in the status of said preselected body muscle from a relaxed state to a flexed state, and vice versa. The system further includes an indicator circuit configured to be communicatively coupled to the pressure sensor and, drawing from a power supply, configured to provide one or more indications of the status of said preselected body muscle, including the relaxed state and the flexed state.

According to other embodiments, the indicator circuit further includes one or more light emitting diodes (LEDs), the indicator circuit is configured to indicate the relaxed state by illuminating at least one of the one or more LEDs in a first color, and the indicator circuit is configured to indicate the flexed state by illuminating at least one of the one or more LEDs in a second color that is different than the first color.

In some embodiments, the indicator circuit further includes a visual indicator of an adequate connection to the power supply, a proper fastening of the strap, or the status of said preselected body muscle.

In other embodiments, the indicator circuit is further configured to transmit or communicate pressure readings to an external device such as a computer equipped with software allowing for processing of the data or a commercially available audio interface that can correlate data from an instrument and data received form the device.

In other embodiments, the strap may include a haptic feedback module, vibrating motor, or a buzzer.

In still other embodiments, the vibration of the motor can be processed by software to generate a sonic waveform indicating or correlating with the pressure detected by the pressure sensor. This data is transmitted to a receiver, then to an audio interface where the amplitude and frequency of the force are recorded. In this manner the user can immediately track the pressure detected by the pressure sensor directly recorded alongside data from a task, such as playing music, to identify where pressures exceed an acceptable limit.

According to many embodiments, the system further includes a memory configured to record at least one of instances when the preselected body muscle transitions from one state to another.

In other embodiments, the strap further includes a mark, visible on the side of the strap opposite the one side, to assist a user with proper positioning or alignment of the pressure sensor relative to an anatomical feature.

In yet another embodiment, the anatomical feature includes an elbow lateral epicondyle, an elbow medial epicondyle, a bicep, a calf, a thigh muscle, or other muscle or muscle group in the body.

In many other embodiments, the indicator circuit is coupled to or integrated with the strap.

According to many embodiments, the indicator circuit is further configured to indicate that a strap has been fastened at a proper level of tightness.

In yet another embodiment, the preselected body muscle is selected from a group consisting of a extensor carpi radialis brevis, supinator, extensor digitorum, extensor digiti minimi, extensor carpi ulnaris, common flexor tendon, flexor carpi radialis, flexor carpi ulnaris, flexor digitorum superficialis and palmaris longus.

According to other embodiments, a flexed state arises from a muscle contraction, a wrist extension, or a wrist flexion.

According to many embodiments, the external device comprises a pressure gauge or readout, a computer, a smart watch, or a smart phone.

According to a second aspect, a pressure sensing brace for monitoring a preselected body muscle includes a strap having a sensing portion and a latching portion, the latching portion structured to removably fasten the strap about a preselected body muscle and to position the sensing portion on or proximate to the preselected body muscle. The brace further includes a pressure sensor coupled to or integrated with the sensing portion of the strap, the pressure sensor configured to sense at least a first pressure corresponding to a first preselected body muscle state and a second pressure corresponding to a second preselected body muscle state. The brace also includes a sensing circuit communicatively coupled to the pressure sensor and configured to generate an alert responsive to the pressure sensor sensing at least one of the first pressure or the second pressure.

According to still further embodiments, the brace can include, integrate or otherwise incorporate other forms of sensors to collect different forms of data as needed. For example, it can be desirable when treating a patient with a proprioceptive awareness deficit to monitor movement and/or position instead of or in addition to pressure. The brace can include a variety of different sensors including accelerometers or a Hall effect sensor. Various embodiments can integrate additional sensors into the brace to correlate data from the pressure sensor with other data including, but not limited to temperature, proximity, position, motion, force, contact and level sensors as desired.

According to other embodiments, the brace further includes an alignment indicator configured to provide a visual indication to a user to place the brace in a position such that the pressure sensor is correctly aligned with the preselected body muscle.

In other embodiments, the alert is a visual alert.

In many embodiments, the sensing circuit is configured to provide a first alert responsive to the pressure sensor sensing the first pressure and a second alert responsive to the pressure sensor sensing the second pressure.

According to some embodiments, the sensing circuit is coupled to or integrated with the brace.

In many other embodiments, the second pressure is greater than the first pressure.

According to a third aspect, a brace includes a strap having two sides, one side configured to be positioned over and removably fastened to a body surface directly over a preselected body muscle. The brace further includes a pressure sensor integrated with the strap and configured to detect changes in pressure applied to said one side of said strap in response to a change in the status of said preselected body muscle from a relaxed state to a flexed state, and vice versa. The brace further includes a mark, visible on the side of the strap opposite the one side, to assist a user with proper positioning or alignment of the pressure sensor relative to an anatomical feature, wherein the pressure sensor is configured to be communicatively coupled to a pressure indicator.

According to some embodiments, the preselected body muscle includes a forearm, upper arm, or leg.

According to a fourth aspect, a method of monitoring a user's forearm muscle strain, the method includes providing a brace comprising a strap having two sides and structured to be secured about a circumference of a forearm, wherein the strap includes a pressure sensor configured to monitor a state of a forearm muscle and provide an indication corresponding to the state of the forearm muscle. The method further includes providing a first indication when the forearm muscle is in a relaxed state and providing a second indication when the forearm muscle transitions from the relaxed state to a strained state.

According to many embodiments, the strap and pressure sensor are configured to monitor the state of the forearm muscle when the user is playing a musical instrument.

In other embodiments, the second indication alerts the user that the musical instrument is being played with an undesirable forearm muscle strain or wrist position.

According to some embodiments, the strap and pressure sensor are configured to monitor the state of the forearm muscle when the user is playing a sport involving holding a piece of sporting equipment, such as a tennis racket, baseball bat, ball, or golf club, and wherein the second indication alerts the user that the sporting equipment is being held in an undesirable manner.

In many embodiments, a side of the strap, which is visible to the user, bears a mark to assist the user with proper positioning or alignment of the pressure sensor relative to an anatomical feature.

According to other embodiments, the method further includes providing a tightness indicator configured to indicate to the user that the strap has been properly tightened to monitor the state of the forearm muscle.

In some embodiments, the method relieves one or more symptoms of lateral epicondylitis or medial epicondylitis.

In some embodiments, the brace includes microprocessor control, which can detect movement direction and velocity, as well as gravitational pull. When combined with the pressure sensor mechanism, the brace can measure muscle tension versus relaxation and also movement versus stillness. This movement sensor can also be used to develop time-based relationships of tension and movement. For example, a golfer's swing can be graphed showing grip tension changes throughout all stages of their golf swing such that “problem areas” can be identified. In some embodiments, the movement sensor is an accelerometer. In other embodiments, the movement sensor is a 3-axis accelerometer and in still further embodiments, it is a MIMS accelerometer.

Many embodiments of the brace can be used in practice and research (e.g., medical, behavioral, neurophysiological), where the tension of a body part and posture should be measured with minimal constraint or influence from the measuring devices on the target body part. For example, when measuring proprioceptive ability of the hands and fingers, the brace can measure level of relaxation of the hand, and fingers, the brace can measure the level of relaxation of the hand/wrist/forearm, so a base proprioceptive awareness can be measured under minimal hand and finger tension, as a proxy measured by the brace through monitoring forearm tension, deflection, and circumference. The movement sensing capability described above further allows the brace to monitor, alert and record any movement in three-dimensional space.

The brace can also be used in investigation of malingering and cases were patient intent and effort can be interpreted by changes in tension, shape and other physical properties of a body part or muscle group which should be recruited in valid attempts of physical performance of the reportedly injured body part. When combined with other objective and quantifiable physical measures (e.g., optic sensing and recording) and neurophysiological measures (e.g., cognitive activation/Brain wave patterns and GSR) a convergent validation of the authenticity of the injury and/or the patient's effort level can be assessed in an objective, quantifiable and legally defensible fashion.

### BRIEF DESCRIPTION OF THE DRAWINGS

The disclosure will become more fully understood from the following detailed description, taken in conjunction with the accompanying figures, wherein like reference numerals refer to like elements, in which:

FIG. 1 is a perspective views of an embodiment of a brace worn on about a forearm muscle in a relaxed state.

FIGS. 2A and 2B are perspective views of an embodiment of a brace worn on about a forearm muscle in a flexed state.

FIG. 3 is a top plan view of an embodiment of a brace.

FIG. 4 is a side cutaway view of the pressure sensor, power supply, and various indications of the brace of FIG. 3.

FIG. 5 is a block diagram of the pressure monitoring and alerting system.

FIG. 6 is a circuit diagram of the circuit of the brace of FIG. 3.

FIG. 7 is a top plan view of an embodiment of a rechargeable brace.

FIG. 8 is a top plan view of an embodiment of a connected brace.

FIG. 9 is a top plan view of an embodiment of a display brace.

FIG. 10 is a top plan view of an embodiment of a vibrating brace.

FIG. 11 is a top plan view of an embodiment of a motion sensing brace.

FIG. 12 is a view of a user interface displayed on an external device.

FIG. 13 is a flow chart of how data collected by the brace is accessed by trained personnel.

FIG. 14 is a decision tree flow chart of how to properly don the brace.

FIG. 15 depict the brace being worn during various exercises and activities, particularly throwing a baseball.

FIG. 16 depicts the brace being worn on or about a user's calf.

FIG. 17 depicts the brace being worn on or about a user's thigh.

FIG. 18 depicts the brace being worn on or about a user's bicep.

FIG. 19 depicts the brace being worn on or about a user's chest.

FIG. 20 depicts the brace being worn on or about user's jaw.

FIG. 21 is a flow diagram depicting a method of using the brace to improve a user's ability to play the guitar.

The drawings are not necessarily to scale, and certain features may be shown exaggerated in scale or in somewhat schematic form in the interest of clarity and conciseness.

### DETAILED DESCRIPTION

The brace 100 herein disclosed provides an apparatus and method of measuring the level of strain in a body part during a particular activity or set of activities. As a result, the brace 100 can be used to help screen applicants for a particular task or job based upon how well they perform the task(s) without producing too much strain in a particular body part. In this manner, the brace 100 can not only identify someone that is adept at a particular task but can be used to train someone less adept at the same task to perform it better, i.e., without as much strain on their body. The brace can be used to assess a variety of tasks and movements in this manner including, but not limited to weightlifting, lifting in general, and detecting unintentional behaviors such as jaw clenching and/or teeth grinding. Furthermore, the brace 100 may be useful in treating and/or recovering from certain medical conditions that may affect a person's proprioceptive awareness. For example, patients with traumatic brain injuries and other forms of brain damage may benefit from receiving feedback from certain portions of their body that they may not otherwise receive because of their medical condition. The practical applications for methods of using the brace 100 are numerous and the below description is merely meant to illustrate various embodiment of the brace 100 as well as a variety of methods of employing the brace 100.

The brace 100 provides a training device that aids to prevent injury and improves performance of tasks where a firm grip is necessary to perform the task, but an excessive grip can result in reduced task performance and injury. As a prominent example, epicondylitis is a frequent injury for musicians (e.g., guitarists). A difficult concept and skill to command in guitar playing is to relax the fretting hand while practicing intensely. The player's grip should be only tight enough such that the strings play clearly when depressed to make contact with the frets. However, given the rigors, complexity, and stress of learning multiple chording and single note techniques, students tend to overtighten their grip. Even guitarists who have previously learned to play with a very relaxed and healthy technique will tend to grip too tightly under stress. This excessive grip not only leads to injury but decreases the player's ability to play in direct relationship to the over-gripping of the instrument. When properly worn, the brace 100 provides immediate feedback to the player when they are gripping too tightly, so that the player can relax their grip to a level that reduces unnecessary tendon stress or strain.

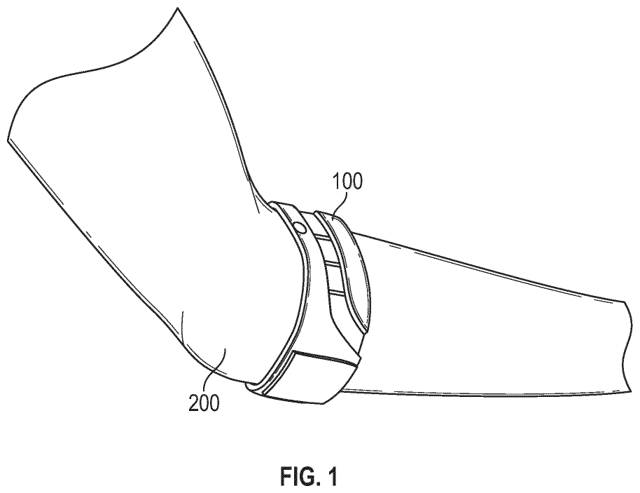

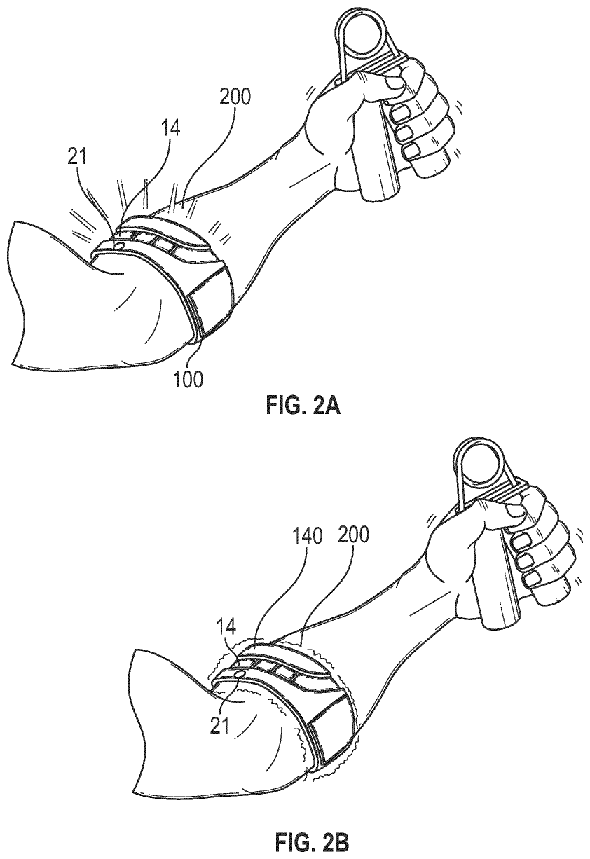

Reference is now made to FIGS. 1-2B, which depicts a brace 100 or a vibrating brace 140 worn on a forearm muscle 200 in a relaxed state (FIG. 1) and in a flexed state (FIGS. 2A-2B). When the brace 100 is worn with the proper tension in the strap 10, a relaxed or first indication 16 is activated. As the forearm muscle 200 contracts or flexes to a degree that can cause lateral or medial epicondylitis, a flexed or second indication 14 is activated to alert the wearer that their forearm muscle 200 is causing excessive stress or strain to the tendons of the forearm muscle 200 and that the wearer should relax the forearm muscle 200. As shown in FIG. 2A, the flexed or second indication 14 can be a visual light that is illuminated on the brace 100, as will be discussed in further detail below. As shown in FIG. 2B, the flexed or second indication 14 can be a tactile sensation generated by a haptic feedback module 40 activating to vibrate the vibrating brace 140, as will be discussed in further detail below. It should be understood, that while the brace 100 is depicted about a forearm muscle 200, the brace 100 may also be used with other muscle groups. For example, the brace 100 may be used to monitor the extensor carpi radialis brevis, supinator, extensor digitorum, extensor digiti minimi, extensor carpi ulnaris, common flexor tendon, flexor carpi radialis, flexor carpi ulnaris, flexor digitorum superficialis, palmaris longus, gastrocnemius, soleus, pectineus, sartorius, and/or quadriceps femoris, biceps brachii, triceps brachii, the deltoid, the brachioradialis or any other muscle or muscle group which the brace 100 can be fitted around, on, or about.

Continuing with reference to FIGS. 2A-2B, the strap 10 includes a mark 21 thereon to aid the wearer to properly align the pressure sensor 24 with the forearm muscle 200. The mark 21 may be a visual mark they the wearer aligns with a particular anatomical feature before tightening the strap 10 to the proper tension. The mark 21 may also be a tactile bump or ridge that the wearer aligns with a particular anatomical feature before tightening the strap 10 to the proper tension. As shown in FIG. 2, the mark 21 is positioned such that when the mark 21 is aligned with the crux of the wearer's elbow, then the pressure sensor 24 is optimally positioned about the forearm muscle 200.

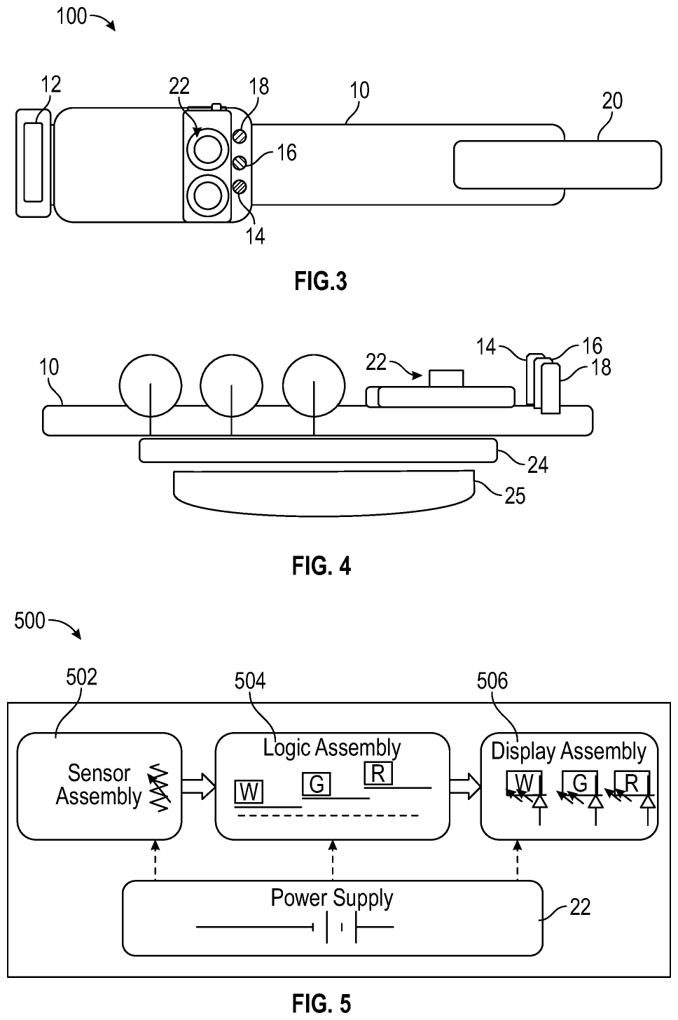

Referring now to FIGS. 3 and 4, which respectively depict a top plan view and a side plan view of the brace 100. The brace 100 includes a strap 10, a tension ring 12, a fastening member 20, a power supply 22, a pressure sensor 24, a flexed indicator 14, a relaxed indicator 16, and a power indicator 18. The strap 10 is circumferentially worn around a wearer's forearm muscle 200 such that the pressure sensor 24 is positioned to optimally measure the forearm muscle 200 flexing to a degree as to potentially cause elbow tendinopathy.

The strap 10 is configured to be worn circumferentially around a pre-selected muscle group, such as, for example, a forearm muscle 200. The strap 10 is connected to the fastening member 20 on one end of the strap 10 and the tension ring 12 on the other end of the strap 10. The strap 10 also includes a fastening portion along a surface of the strap 10 such that the fastening member 20 can be secured to, fastened to, or otherwise removably coupled to the fastening portion of the strap 10. The fastening portion of the strap 10 and the fastening member 20 can each be part of a hook and loop fastening system, where the fastening portion is made up of the loops and the fastening member 20 is made up of the hooks. When the fastening member 20 interlocks with the fastening portion of the strap 10, the circumference of the strap 10 is fixed.

The tension ring 12 includes a through-hole or opening to allow the fastening member 20 to pass through. The tension of the strap 10 may be adjusted mechanically by the amount of the strap 10 that is pulled through the tension ring 12 and then doubled back against itself and secured with the fastening member 20. In this configuration, a user only needs a single hand to don and tension the brace 100 with a single grasp and pulling motion.

The pressure sensor 24 is located on the side of the strap 10 that makes contact with the wearer when the brace 100 is worn. The pressure sensor 24 is configured to detect the changes in pressure caused by the forearm muscle 200 flexing and thereby circumferentially expanding against the strap 10, which itself maintains a substantially consistent circumference. The interaction between the expanding circumference of wearers forearm muscle 200 against the fixed circumference of the strap 10 creates the pressure that is read by the pressure sensor 24. The pressure sensor 24 is positioned between the strap 10 and the sensor cushion 25. The sensor cushion 25 and the pressure sensor 24 can be integrated into the strap 10 or may be removably coupled to the strap 10 with a plurality of fasteners or any other suitable fastening system. The pressure sensor 24 may be any one of a number of commercially available devices that convert pressure into an electrical signal that can be stored, displayed, read and/or transmitted. In some embodiments, the pressure sensor 24 detects the pressure by utilizing the piezoelectric effect, which certain materials produce an electric current in response to stress, including pressure, turning or twisting.

The indications 14, 16, 18, may be visual indications as shown in FIG. 2A, tactile indications (e.g., vibrator) as shown in FIG. 2B, auditory indications, or any combination thereof. For the embodiment depicted in FIGS. 3-4, the indications 14, 16, and 18 are differently colored LEDs, where the power indication 18 is a white LED, the relaxed indication 16 is a green LED, and the flexed indication 14 is a red LED. In some embodiments, the LED's provide dual functionality as both the logic assembly 504 and as part of the display assembly 506, as discussed in further detail below.

The power indication 18 is configured to activate or light up when the brace 100 is powered on. The relaxed indication 16 is configured to activate or light up when the brace 100 is donned with the proper tension about the muscle 200. The flexed indication 14 is configured to activate or light up when the pressure sensor 24 senses that the muscle 200 has transitioned to a state that may unduly stress or damage the muscle 200 or the tendons of the muscle 200. In some embodiments, the relaxed indication 16 stays activated when the flexed indication 14 is activated.

Reference is now made to FIG. 5, which depicts a pressure monitoring and alerting system 500 utilized by the brace 100. The system 500 includes a pressure sensor assembly 502, a logic assembly 504, a display assembly 506, and a power supply 22. The logic assembly 504 interprets the pressure readings from the pressure sensor assembly 502 as the tension of the strap 10 changes, either from initially donning the brace 100 or while the brace 100 is in use. As such, as the pressure sensor assembly 502 reads ever-increasing pressure, the logic assembly 504 causes the display assembly 506 to activate different indications to the wearer corresponding to different states of the forearm muscle 200, such as a flexed state or a relaxed state. When the brace 100 is properly worn, the display assembly 506 will activate a relaxed indication 16 when the forearm muscle 200 is in the relaxed state and activate a flexed indication 14 when the forearm muscle 200 is in the flexed state.

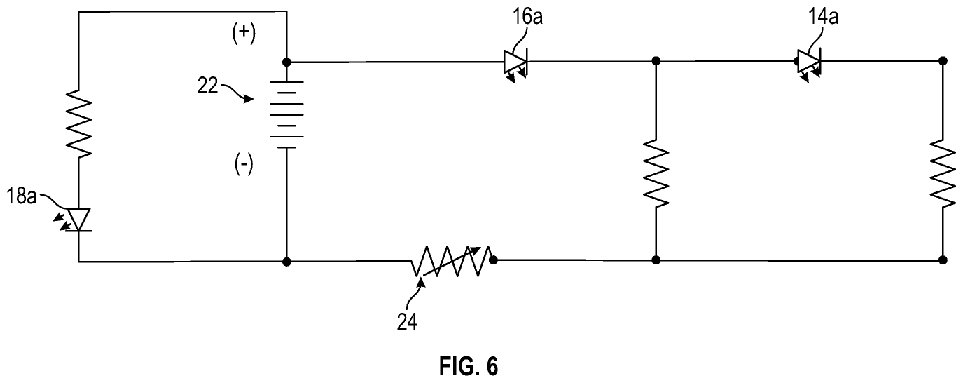

Referring now to FIG. 6, which illustrates an example solid state schematic of the pressure monitoring and alerting system 500 utilized by the brace 100. This type of circuit may be referred to as a divided voltage ladder, with Light Emitting Diode (LED) indication. The power supply 22 includes two batteries connected in series such that the power supply 22 produces 6VDC. Using two batteries in series not only increases finite amount of usage before either battery requires replacement, but also simplifies the logic circuit. Many readily available LEDs have a traditional forward biasing characteristic of roughly 2VDC. As such, each LED drops 2 volts at forward bias and therefore, allows the LEDs to function as both the logic assembly 404 and the indication assembly 406.

As the resistance of the pressure sensor 24 decreases, potential voltage drop across the pressure sensor 24 decreases until the LED 16a achieves forward bias (2VDC), allowing current to flow through the LED 16a. In this sense, the LED 16a is performing both logic and indication functions for determining if the muscle 200 is in the relaxed state. As the resistance of the pressure sensor 24 decreases further, potential voltage drop across the pressure sensor 24 decreases until the LED 14a achieves forward bias (2VDC), allowing current to flow through the LED 14a. In this sense, the LED 14a is performing both logic and indication functions for determining if the muscle 200 is in the flexed state.

The depicted resistors function as current limiters for the LEDs and can be readily replaced with current limiting devices in a Surface Mounted Device miniature component format. Also, the LED 18a is configured to activate when the brace 100 is switched on by closing a single pole switch (not shown). The current to LED 18a is severely limited by a relatively large current limiting resistor to reduce the drain on the power supply 22 and maximize the life of the power supply 22.

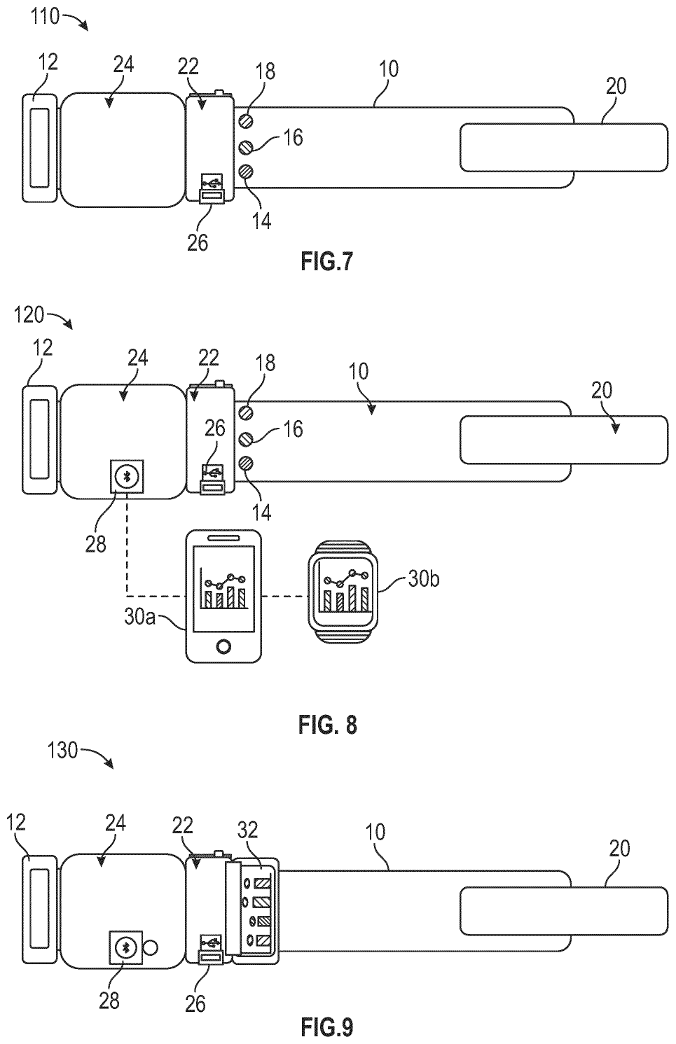

Referring now to FIG. 7, which depicts a top plan view of a recharging brace 110. The recharging brace 110 is an alternate embodiment of the brace 100 and includes all of the components of the brace 100, with the addition of a recharging module 26. Recharging module 26 allows for the power supply 22 to be recharged when the power level becomes low as opposed to being outright replaced. Recharging module 26 can facilitate either wired or wireless charging of the power supply 22. While recharging module 26 is presently depicted as a standard female USB-A connector, alternate configurations may be used. For example, recharging module 26 can be a standard female USB-C connector, a standard qi wireless charging module or any other wireless charging standard, a proprietary connector, or any other suitable connector or module to facilitate charging of the power supply 22.

Reference is now made to FIG. 8, which depicts a top plan view of a connected brace 120. The connected brace 120 is an alternate embodiment of the brace 100 and includes all of the components of the brace 100, with the addition of a communications module 28. The connected brace 120 can also include the recharging module 26 to facilitate recharging of the power supply 22. The communications module 28 is configured to interface with or otherwise be communicatively coupled to the pressure sensor 24 and transmit the pressure that is read by the pressure sensor 24 to an external device 30a/30b. Communications module 28 can also be configured to only transmit the state of the forearm muscle 200 (relaxed, flexed, or other) to the external device 30a/30b. The communications module 28 can also include a memory configured to register and store the state of the forearm muscle 200 or the pressure read by or sensed by the pressure sensor 24 throughout the day. In such embodiments, the communications module 28 can be configured to only transmit the stored data at a single, pre-determined time every day to the external device 30a/30b to reduce the power drain on the power supply 22. The communications module 28 can also be configured to transmit the data in real-time to the external device 30a/30b via a wireless connection, for example, a Bluetooth connection.

Referring now to FIG. 9, which depicts a top plan view of a display brace 130. The display brace 130 is an alternate embodiment of the brace 100 and includes all the components of the brace 100, with the addition of a display 32. The display brace 130 can also include the communications module 28, the recharging module 26, or both. The display 32 is configured to interface with or otherwise be communicatively coupled to the pressure sensor 24 and display either the pressure read by the pressure sensor 24 or the state of the forearm muscle 200. The display 32 is similarly configured to provide the various indications 14, 16, and 18, corresponding to the current state of the forearm muscle 200. For example, when display 32 provides the flexed indication 14, the display 32 may be configured to display an all-red screen to alert the wearer that their forearm muscle 200 has transitioned into the flexed state.

According to some embodiments, the display 32 is a touch sensitive display which allows for the wearer to interact with the display 32. The wearer can alter various settings of the display 32, alter what or how information is depicted on the display 32, or a combination thereof.

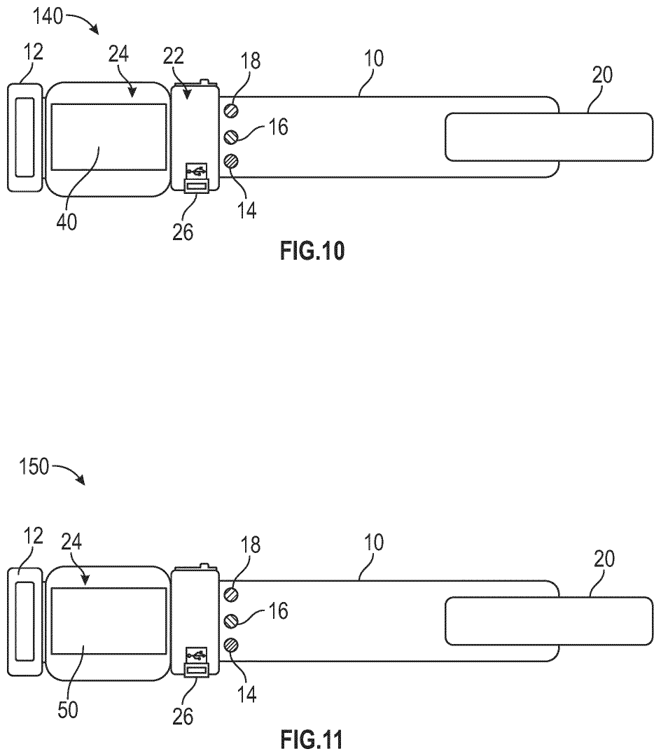

Referring now to FIG. 10, which depicts a top plan view of a vibrating brace 140. The vibrating brace 140 is an alternate embodiment of the brace 100 and includes all the components of the brace 100, with the addition of a haptic feedback module 40. The vibrating brace 140 can also include the recharging module 26 to facilitate recharging of the power supply 22 and the communications module 28 to transmit the pressure that is read by the pressure sensor 24 to the external device 30a/30b. The vibrating brace 140 can also include a display 32 as discussed above in reference to the display brace 130. The haptic feedback module 40 is configured to alert the user of the state of their forearm muscle 200 (or any other muscle group) as the pressure sensor 24 senses varying pressures in the forearm muscle 200 throughout the day. As the vibrating brace 140 detects that a user's muscle group has transitioned to a flexed state, the haptic feedback module 40 will activate and vibrate to alert the user. The haptic feedback module 40 can also be configured to provide a series of vibrations to alert the user that the muscle group has been in a flexed state for an extended period of time. Such period of time can be set or otherwise determined by the user. In other embodiments, the haptic feedback module 40 is configured to produce vibrations with progressively higher or lower frequencies as the pressure detected by the pressure sensor 24 increases or decreases. In certain embodiments, the haptic feedback module 40 can be operably connected to an external device 30a/30b that can generate and/or display a sonic waveform or other visual representation of the frequency of the vibrations of the haptic feedback module 40. This allows the user to track the strain in their muscle 200 while they are performing a particular activity.

Referring now to FIG. 11, which depicts a top plan view of a motion sensing brace 150. The motion sensing brace 150 is an alternate embodiment of the brace 100 and includes all of the components of the brace 100, with the addition of a motion sensor 50. The motion sensing brace 150 can also include the recharging module 26 to facilitate recharging of the power supply 22 and the communications module 28 to transmit the pressure that is read by the pressure sensor 24 to the external device 30a/30b. The motion sensing brace 150 can also include a display 32 as discussed above in reference to the display brace 130. The motion sensor 50 is configured to monitor the position and movement of a user's body part. Such monitoring may be useful in treating proprioception disorders or other medical conditions that affect a patient's proprioceptive awareness. The motion sensor 50 can be any suitable device that can detect movement, including various types of accelerometers, such as multiple axis accelerometers or MIMS accelerometers. In such embodiments, the motion sensor 50 is integrated into the motion sensing brace 150 and is operably connected to the power supply 22 of the motion sensing brace 150.

The motion sensing brace 150 may be useful in treating disorders that affect proprioception such as sensory processing disorders, brain injuries, multiple sclerosis, Lou Gehrig's disease and/or other disorders caused by brain damage. The motion sensing brace 150 can provide feedback to guide a user who struggles with proprioceptive awareness, which the user can then use to adjust the way they perform certain tasks. For example, if the user struggles to write without using an excess of force, then the motion sensing brace 150 can be used to provide feedback useful in addressing such a problem.

The communications module 28 can also be configured to communicate or transmit other data collected by or sensed by other sensors connected to or communicatively coupled to the connected brace 120, the display brace 130, the vibrating brace 140, and/or the motion sensing brace 150.

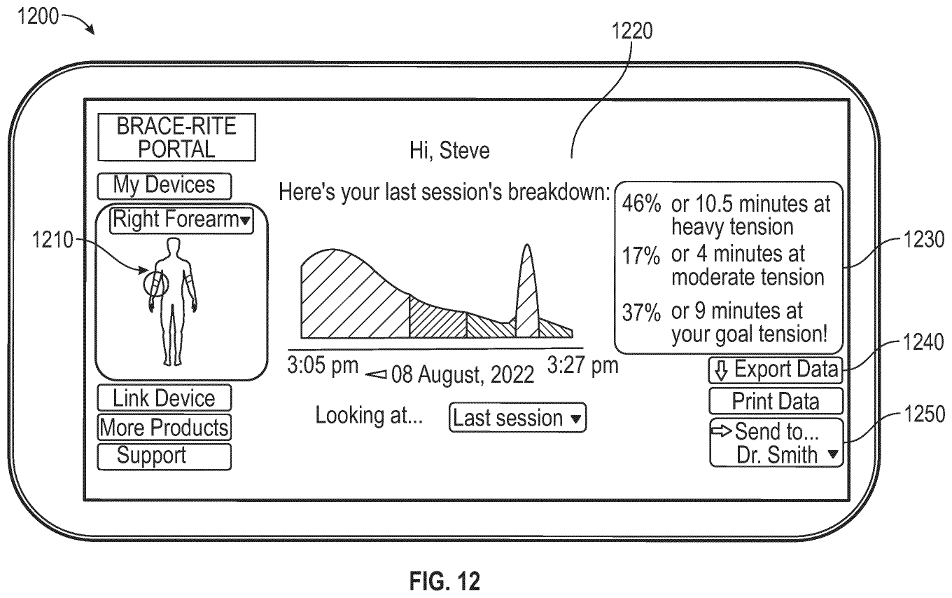

Reference is now made to FIG. 12, which depicts a user interface 1200. The user interface 1200 allows for the wearer to analyze their performance over the course of a day, week, month, or year. The user interface 1200 includes an identification module 1210, a graphical display module 1220, a percentage display module 1230, an export data module 1240, and a pre-populated export module 1250. The identification module 1210 depicts which muscle 200 the data displayed corresponds to. For example, the identification module 1210 can identify the right forearm, the left forearm, or any other muscle group that the brace 100 is utilized thereon. The graphical display module 1220 depicts the varying pressures that the pressure sensor 24 read throughout the session in a graphical or other visual format. The percentage display module 1230 displays what percentage of time that the muscle group spent in each particular state throughout the session. The export data module 1240 allows the user to export the data displayed on the user interface 1200 to a separate program or server for storage or further analysis. The pre-populated export module 1250 allows the user to export the data directly to a particular entity or person, such as a trainer or healthcare professional so that the data can be directly reviewed and analyzed by the trainer or healthcare professional.

The user interface 1200 displays the amount of time 1230 that the wearer's forearm muscle 200 spent in each state over the course of the day. In this manner, the wearer can determine which activities they were performing when their forearm muscle 200 was in the flexed state and focus on relaxing their grip the next time the wearer performs those activities. In this manner, the brace 100 facilitates providing immediate feedback to the wearer in the form of the relaxed indication 16 or the flexed indication 14 while a task is currently being performed by the wearer while simultaneously providing data that the wearer can review after a task has been performed.

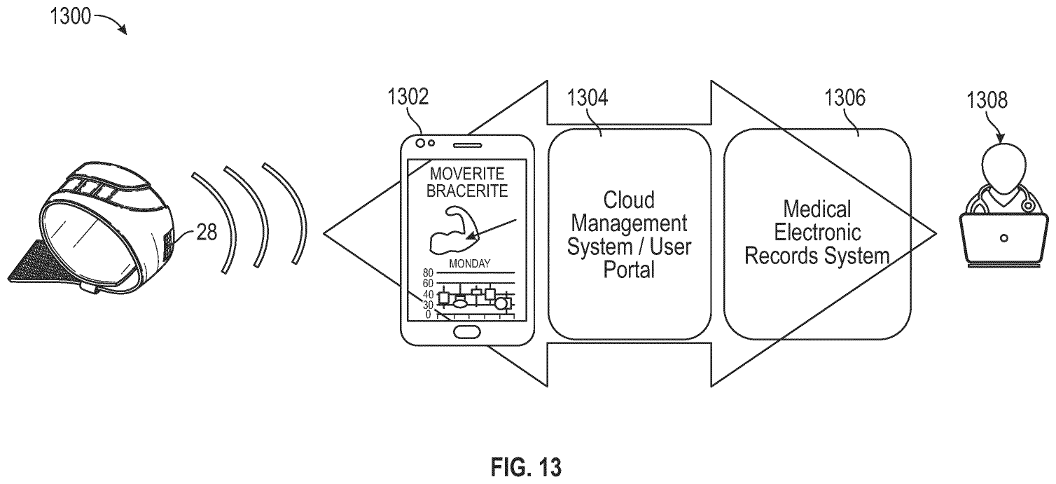

Referring now to FIG. 13, which depicts a flow chart of the flow of the user's data 1300 where the data transmitted by the communications module 28 is first transmitted to an external web connected device 1302. The external web connected device 1302 then transmits the user data to a HIPAA compliant cloud-based solution 1304 that interfaces with a variety of Electronic Medical Record and Educational and Training systems 1106 that is accessible by various health or training professionals 1308. The health or training professionals 1308 can be medical practitioners, educators, teachers, or trainers. The health or training professionals 1308 can then use the data for training, medical care, and associated research efforts.

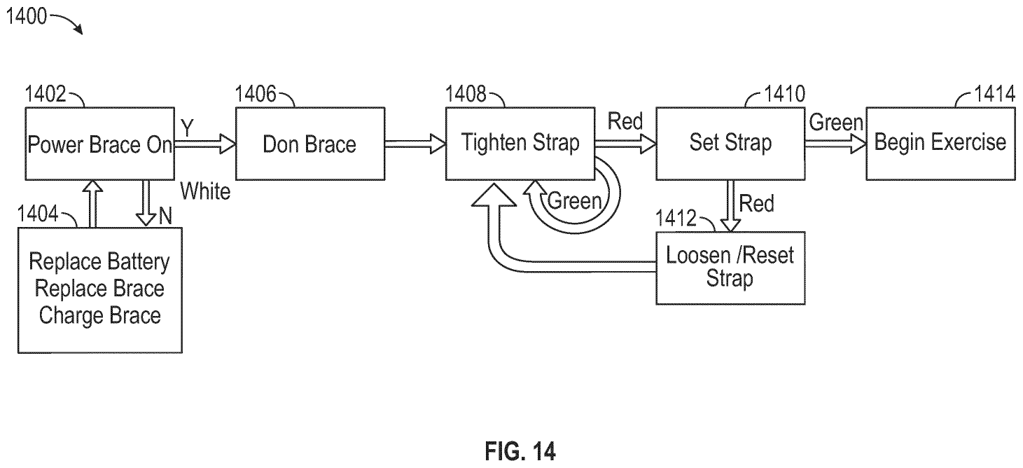

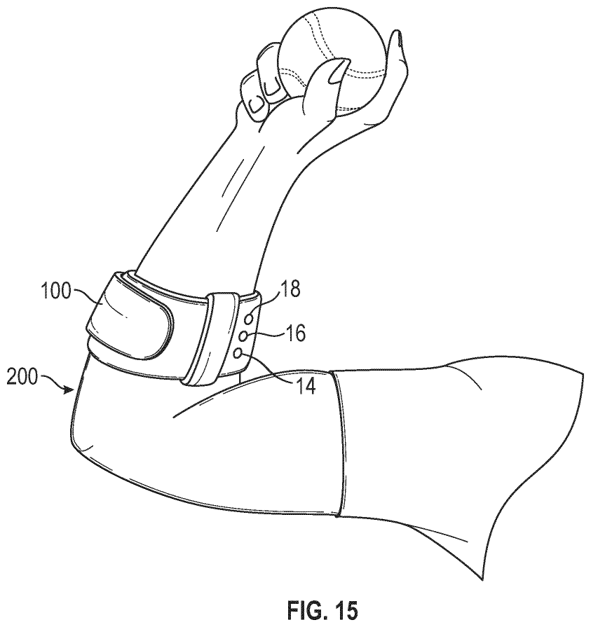

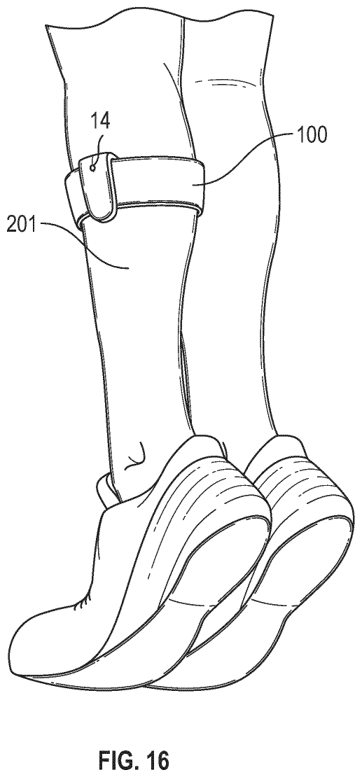

Reference is now made to FIG. 14, which depicts an exemplary flow chart 1400 for properly donning the brace 100. To don the brace 100, a user must first power the brace 100 on 1402, if the brace 100 does not have power, the user must either change out the power supply 22 or charge the power supply 22 at step 1404. The user then loosely positions the strap 10 about the circumference of their forearm muscle 200 at step 1406. The user then must apply the proper tension to the strap 10, so at step 1408 the user tightens the strap 10 until the relaxed indicator 16 is activated and continues tightening the strap 10 until the flexed indicator 14 is activated. The user then sets the tension in the strap 10 by securing the fastening member 20 to the strap 10 at step 1410. If, after the tension in the strap 10 is set in step 1410 the flexed indicator 14 is still activated after step 1410, then the strap 10 is set too tight and the user must relax the tension in the strap 10 until only the relaxed indicator 16 is activated 1412. If, after the tension in the strap 10 is set in step 1410, the relaxed indicator 16 is activated then the strap 10 is set to the proper tension and the wearer may begin their exercise or activity 1414.

Referring now to FIG. 15, which depict the brace 100 being worn while the wearer is participating in during exercises or sports. For example, FIG. 15 depicts a user wearing the brace 100 while throwing a baseball. It should be understood that the brace 100 can be used around or about various different muscles and muscle groups while the user is engaged in various exercise related or otherwise common activities that the user goes about in their daily life. For example, the brace 100 can be implemented by a user holding and using a golf club, holding and using a tennis racket, and/or holding and playing a guitar. Other examples include a user wearing the brace 100 while the user is performing everyday tasks such as carrying a suitcase, swinging a hammer, operating a jackhammer, or exercising their forearm.

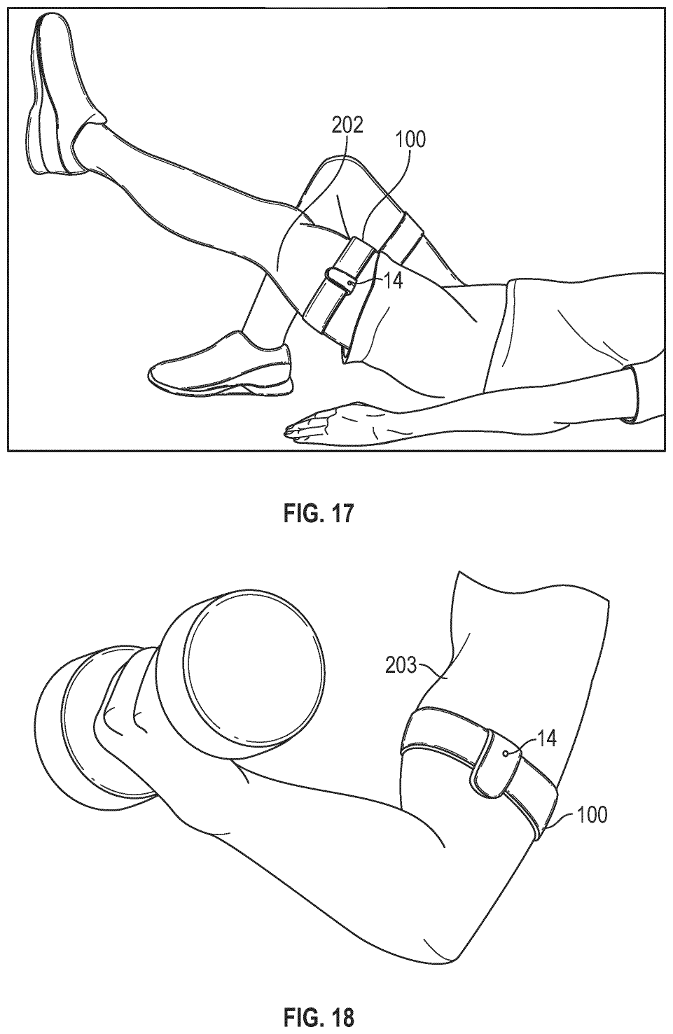

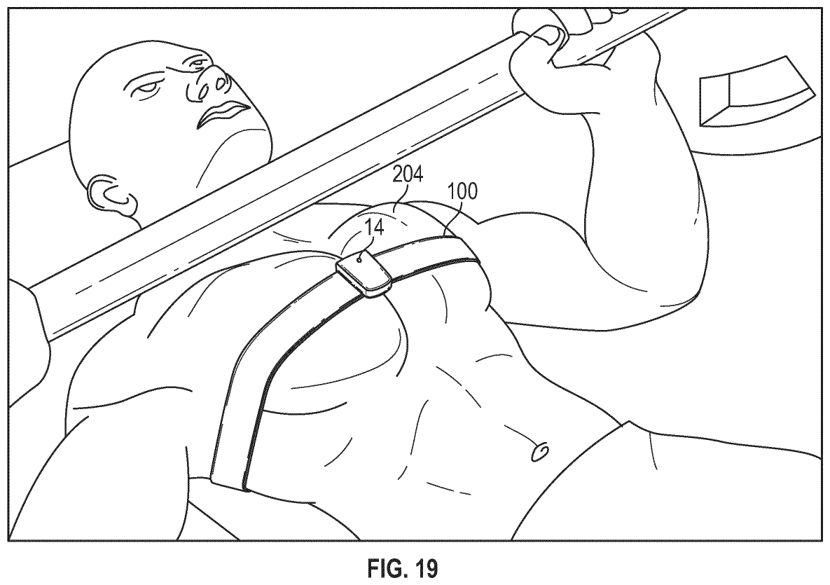

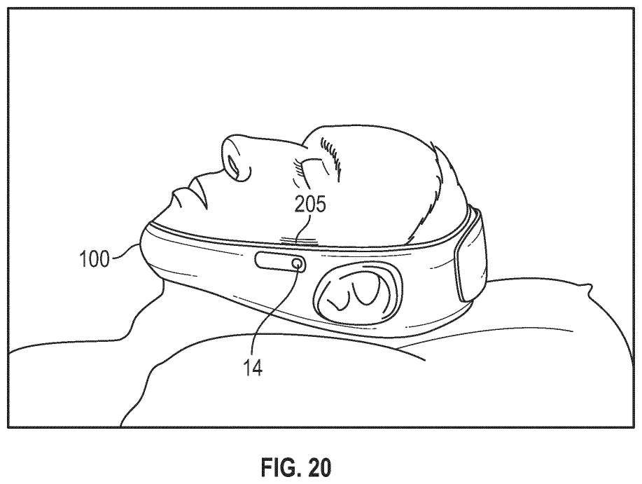

Referring now to FIGS. 16-20, which collectively depict the brace 100 being worn on body parts other than the forearm. For example, FIG. 16 depicts the brace 100 being worn around a user's calf 201 while the user is performing calf raises. Wearing the brace 100 in this manner allows a user to measure or monitor how much they are straining the muscles in their calves 201 while performing exercises such as calf raises. Similarly, FIG. 17 depicts the brace 100 being worn on or about a user's thigh 202 while the user is performing leg exercises. FIG. 18 depicts the brace 100 being worn on or about a user's bicep 203 the user is performing bicep curls. FIG. 19 depicts the brace 100 being worn on or about a user's chest 204. In this manner, the brace 100 can provide feedback to a user regarding the amount of strain placed on their chest 204 while performing weighted chest exercises. FIG. 20 depicts a user wearing the brace 100 on or about their jaw 205. Worn in this configuration, the brace 100 can alert a user when they are grinding their teeth or otherwise tensing their jaw muscles 205. In this manner, the brace 100 can detect behaviors associated with conditions such as bruxism by detecting when and to what degree the user tenses the muscles in their face and/or jaw 205. The brace 100 can be used to detect when the user is straining more than expected, for example, when they perform the exercise improperly.

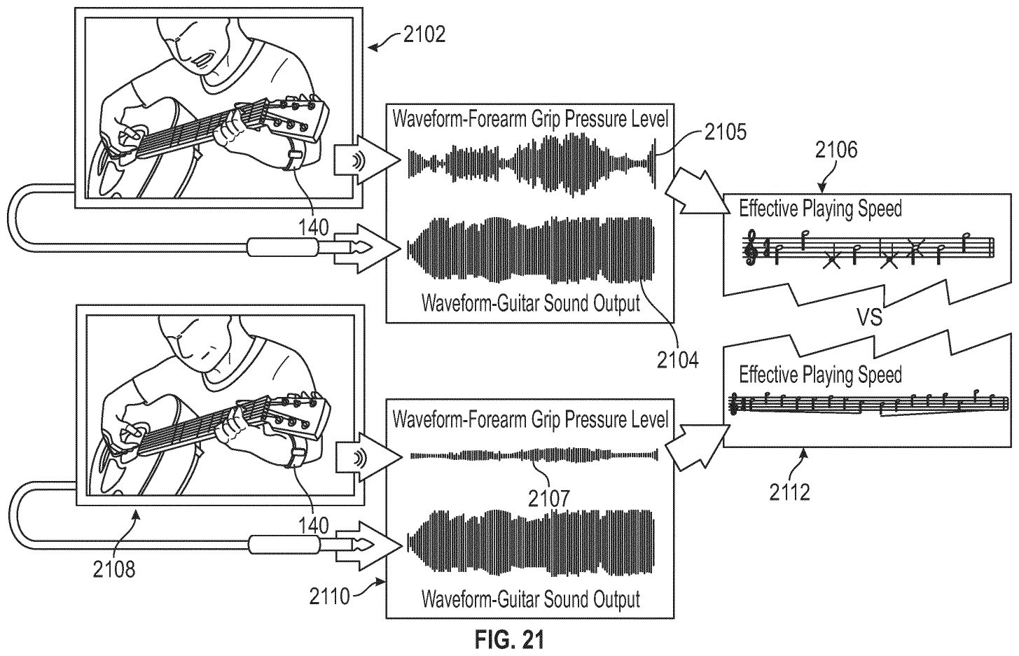

Referring now to FIG. 21 which depicts another embodiment of the brace 140 being worn by a user while the user plays a guitar with a strained forearm muscle 2102 and a relaxed forearm muscle 2108. For example, a user who plays the guitar with excess tension or strain in their forearm 2102 can have the tension waveform 2105 produced by the vibrating brace 140 displayed alongside a waveform produced by sound of the user's guitar 2104. The user's effective playing speed 2106 with the excess muscle tension is also shown. The user can visually identify certain musical notes or series of musical notes that the user is playing that produces larger pressure readings. In this manner, the guitarist can visualize the measurement of the tension in their arm side-by-side with the music being played to determine where they are tensing up too much while playing that piece. The user may then incorporate said feedback and practice to reduce the strain in their muscle 200 while playing 2108. Playing the guitar with reduced strain 2108 would produce a feedback display 2110 which shows the tension waveform 2107 produced by the vibrating brace 140 to have amplitudes which are lower than tension waveform 2105, which indicates that the user is performing the task with less strain in their muscle 200 than before. This reduced strain results in a faster playing speed 2112 for the user.

The brace 100 can be implemented with a variety of applied circuitry which can be designed to minimize the overall part count and minimize the thermal and electromagnetic field emissions for low cost, disposable, or submersible embodiments of the brace 100. The brace 100 can also be implemented with more complex and higher functioning devices which can record and transmit diagnostic information using a wired or wireless communications module 28. The brace 100 can be used to evaluate candidates for a particular task and assess how well they perform that task. As a result, the brace 100 can be used to select candidates for certain tasks and/or jobs for which such candidates are naturally more adept at performing. Further, the brace 100 can be used to measure the effort level of someone performing a task or set of tasks to verify if said person is putting the appropriate level of effort into a task, i.e., the brace 100 can be used to detect malingering. In addition, the feedback provided by the brace 100 can be used to train an individual to better perform a task.

Regardless of where the brace 100 is used or on what part of the body it is placed, the brace 100 provides feedback on the amount of strain being produced in a particular body part, muscle, or muscle group. The brace 100 can be used in any number of contexts to improve performance by monitoring the amount of strain in a particular body part during a movement and using that data to modify the manner in which the movement is performed. For example, the brace 100 may be used by an athlete monitoring the recovery of an injured muscle.

Although embodiments of a brace 100 apparatus and method have been described in detail, those skilled in the art will also recognize that various substitutions and modifications may be made without departing from the scope and spirit of the appended claims.

In the foregoing description of certain embodiments, specific terminology has been resorted to for the sake of clarity. However, the disclosure is not intended to be limited to the specific terms so selected, and it is to be understood that each specific term includes other technical equivalents which operate in a similar manner to accomplish a similar technical purpose. Terms such as “left” and “right,” “front” and “rear,” “above” and “below” and the like are used as words of convenience to provide reference points and are not to be construed as limiting terms.

In this specification, the word “comprising” is to be understood in its “open” sense, that is, in the sense of “including,” and thus not limited to its “closed” sense, that is the sense of “consisting only of.” A corresponding meaning is to be attributed to the corresponding words “comprise,” “comprised” and “comprises” where they appear.

In addition, the foregoing describes some embodiments of the disclosure, and alterations, modifications, additions and/or changes can be made thereto without departing from the scope and spirit of the disclosed embodiments, the embodiments being illustrative and not restrictive.

Furthermore, the disclosure is not to be limited to the illustrated implementations, but to the contrary, is intended to cover various modifications and equivalent arrangements included within the spirit and scope of the disclosure. Also, the various embodiments described above may be implemented in conjunction with other embodiments, e.g., aspects of one embodiment may be combined with aspects of another embodiment to realize yet other embodiments. Further, each independent feature or component of any given assembly may constitute an additional embodiment.

What is claimed is:

1. A bracing system for indicating a potential strain to a muscle, the bracing system comprising:
a strap having at least a first side and a second side, the first side configured to be positioned over and removably fastened to a body surface directly over a preselected body muscle;
a pressure sensor coupled to the strap and configured to detect changes in pressure applied to the first side of the strap in response to a change in a status of the preselected body muscle from a relaxed state to a flexed state, and vice versa; and
an indicator circuit communicatively coupled to the pressure sensor and drawing power from a power source, the indicator circuit configured to provide one or more indications of the status of said preselected body muscle, including the relaxed state and the flexed state.

2. The bracing system of claim 1, wherein the pressure sensor is integral to the strap.

3. The bracing system of claim 1, further comprising a motion sensor coupled to the strap and configured to detect a movement of the preselected body muscle.

4. The bracing system of claim 3, wherein the motion sensor is an accelerometer.

5. The bracing system of claim 1, further comprising a display communicatively coupled to the pressure sensor, wherein the display is configured to display the pressure detected by the pressure sensor.

6. The bracing system of claim 1, wherein the indicator circuit includes one or more light emitting diodes (LEDs), the indicator circuit is configured to indicate the relaxed state by illuminating at least one of the one or more LEDs in a first color, and the indicator circuit is configured to indicate the flexed state by illuminating at least one of the one or more LEDs in a second color that is different than the first color.

7. The bracing system of claim 1, further comprising a communication module communicatively coupled to the pressure sensor and is configured to transmit or communicate the pressure sensed by the pressure sensor to an external device.

8. The bracing system of claim 1, further comprising a memory configured to record at least one of instances when the preselected body muscle transitions from the relaxed state to the flexed state, or vice versa.

9. The bracing system of claim 1, wherein the strap further includes a mark positioned about the second side, wherein the mark corresponds to an anatomical feature of a user such that when the mark is aligned with the anatomical feature, the pressure sensor is aligned over the preselected body muscle.

10. The bracing system of claim 1, wherein the indicator circuit is further configured to transmit or communicate the pressure sensed by the pressure sensor to an external device.

11. A pressure sensing brace for monitoring a preselected body muscle, the pressure sensing brace comprising:
a strap having a sensing portion and a latching portion, the latching portion structured to removably fasten the strap about a circumference of the preselected body muscle and configured to position the sensing portion on or proximate to the preselected body muscle;
a pressure sensor coupled to or integrated with the sensing portion of the strap, the pressure sensor configured to sense at least a first pressure corresponding to a first preselected body muscle state and a second pressure corresponding to a second preselected body muscle state; and
a sensing circuit communicatively coupled to the pressure sensor, the sensing circuit configured to generate a first alert responsive to the pressure sensor sensing the first pressure and generate a second alert responsive to the pressure sensor sensing the second pressure.

12. The pressure sensing brace of claim 11, further comprising an alignment indicator configured to provide a visual indication to a user to place the brace in a position such that the pressure sensor is correctly aligned with the preselected body muscle.

13. The pressure sensing brace of claim 11, wherein at least one of the first alert or the second alert is a visual alert.

14. The pressure sensing brace of claim 11, wherein the second pressure is greater than the first pressure.

15. The pressure sensing brace of claim 11, further comprising a communication module communicatively coupled to the pressure sensor and is configured to transmit or communicate pressure readings to an external device.

16. The pressure sensing brace of claim 15, wherein the external device includes a pressure gauge, a smart watch, or a smart phone.

17. A method of using a pressure sensing brace, the method comprising:
selecting a body part having a muscle or a muscle group;
providing a brace having a strap with two sides and structured to be secured about a circumference of the body part, wherein the strap includes a pressure sensor configured to monitor a state of the muscle or the muscle group and provide a first indication when the muscle or the muscle group is in a relaxed state and provide a second indication when the muscle or the muscle group transitions from the relaxed state to a flexed state;
positioning the strap about the circumference of the selected body part;
positioning the pressure sensor over the muscle or the muscle group of the selected body part;
adjusting a tension of the strap from a positioning tension to an operating tension; and
securing the strap about the circumference of the selected body part at the operating tension.

18. The method of claim 17, wherein the selected body part is a forearm, chest, upper arm, or jaw.

19. The method of claim 17, wherein providing the brace further comprises a motion sensor that is configured to monitor a movement of the selected body part and is communicatively coupled to a transmitter; and
transmitting data of the movement detected by the motion sensor to an external device.

20. The method of claim 17, further comprising providing a tightness indicator configured to indicate that the tension of the strap is at the operating tension.
